(19) United States
(12) Patent Application Publication
(10) Pub. No.: US 2010/0326431 A1
(43) Pub. Date: Dec. 30, 2010

(54) Aerosolization Device

(75) Inventor: Shan-Yi Yu, Taipei County (TW)

Correspondence Address: SCHMEISER OLSEN & WATTS, 18 E UNIVERSITY DRIVE, SUITE # 101, MESA, AZ, 85201, US

(73) Assignee: HEALTH & LIFE CO., LTD., Taipei County (TW)

(21) Appl. No.: 12/877,307

(22) Filed: Sep. 8, 2010

Related U.S. Application Data
(63) Continuation-in-part of application No. 11/979,665, filed on Nov. 7, 2007.

(51) Int. Cl. A61M 11/00; B05B 3/04; B67D 7/08

(52) U.S. Cl. 128/200.14; 239/102.2; 239/72

(57) ABSTRACT

In an aerosolization device, an oscillating aerosol generator is installed at a position proximate to an opening at the bottom of a liquid container for aerosolizing a supplied liquid in the container and a sensor is provided to detect an oscillating signal variation according to a condition whether or not the liquid in the container is in contact with the oscillating aerosol generator and the oscillating signal variation is sent to a controller for computation, so as to control whether to continue operating or stop operating the aerosol generator. If the aerosol generator is controlled to stop its operation, the controller will drive an alarm device to send out an alarm signal at the same time.

### REFERENCE TO RELATED APPLICATIONS

This application is a continuation-in-part of U.S. patent application Ser. No. 11/979,665 filed on Nov. 7, 2007 and published as U.S. Patent Application Publication No. 20090114737 on May 7, 2009.

The above referenced application, and each document cited or referenced in the above referenced application, are hereby incorporated herein by reference.

### BACKGROUND OF THE INVENTION

1. Field of the Invention

The present invention relates to an aerosolization device, and more particularly to an aerosol medication applying device for steadily supplying a fixed quantity of aerosol medication.

2. Description of the Related Art

In general, a prior art aerosolization device comprises a thin aperture plate coupled to a piezoelectric element for providing an AC voltage to the piezoelectric element to generate a vibration with a fixed frequence, so as to drive the thin aperture plate to vibrate. Like a fluid supplier of a lamp wick that sends an aerosol liquid from a storage the aperture to produce an aerosolization and sprayed out from another side of the thin plate by tapping the liquid in contact with the aperture area.

The aerosolization device is used extensively in different fields such as the liquid aerosolization of perfumes, air fresheners or medication, particularly a medication applying device for applying an aerosol medication. A patient with diseases of respiration can use this aerosolization device to aerosolize a medication and sucks the aerosol medication from a suction opening of the aerosolization device into the patient's bronchus and lung for a treatment or mitigation.

In general, a medical aerosolization device is required to steadily and uniformly supply a fixed quantity of aerosol medication per unit time in order to assure treatment effectiveness.

U.S. Pat. No. 6,679,436 discloses a spray which sprays a liquid medicine from a spray part. When a liquid sensing electrode arranged on the spray part senses the quantity of the liquid stored in a storage part and determines that the quantity of the liquid decreases, a pin part is driven by a solenoid for feeding the liquid medicine from a liquid medicine bottle to the storage part of the spray part through a feed pipe, and the feeding interval is obtained when the liquid medicine is intermittently fed for adjusting power supplied to a piezoelectric element of the spray part so that the feeding interval is constant.

U.S. Pat. No. 6,769,626 discloses an improved device and method for measuring the amount of liquid to be discharged that is present in an apparatus, such as a nebulizer. A first member has holes through which the liquid passes to be nebulized. A second member is spaced from the first member so that a volume is defined in the nebulizer by the area of mutual overlap of the first and second members and the amount of spacing between them. The first and second members are formed to establish a mutual capacitance that reflects the amount of liquid in the volume. A measuring circuit measures the capacitance existing between the first and second members and hence the amount of liquid in the nebulizer. The capacitance measurement can be used to control the supply of liquid into the volume in the nebulizer.

However, in the above conventional aerosolization devices, the supply of liquid medication may be interrupted or exhausted without being observed easily. Under this situation, the expected effect of a patient's medical treatment may be seriously affected. At present, there is no prior art aerosolization device that can provide an effective solution.

### SUMMARY OF THE INVENTION

It is a primary objective of the present invention to provide an aerosolization device that uses a sensor to monitor whether or not a liquid in a container is in contact with an oscillating aerosol generator at anytime. If the supply of the liquid is interrupted or exhausted, the controller will control the oscillating aerosol generator to stop its operation immediately, so as to assure the normal operation of the aerosolization device to uniformly and steadily provide a fixed quantity of aerosol medication to patients.

A secondary objective of the present invention is to provide an aerosolization device, such that when a controller stops the operation of the oscillating aerosol generator, the controller will start an alarm device to send out an alarm signal at the same time to notice a patient to stop using the aerosolization device or refill the medication, so as to normally supply liquid medication to the oscillating aerosol generator.

To achieve the foregoing objective, the aerosolization device comprises: a container for containing a liquid; an oscillating aerosol generator being installed at a position proximate to an opening at the bottom of said container for aerosolizing and spraying said liquid in said container to the outside; a sensor being disposed at a position close to said oscillating aerosol generator at the bottom of said container for detecting an oscillating signal from an oscillating aerosol generator through said liquid, and an oscillating signal variation according to a condition whether or not said liquid in said container is in contact with said oscillating aerosol generator; a controller for controlling the operation status of said oscillating aerosol generator according to said oscillating signal variation detected by said sensor, and an alarm device being electrically connected with said controller and for sending out an alarm signal; wherein said liquid is a medium for delivering said oscillating signal from said oscillating aerosol generator to said sensor.

The above and other objects, features and advantages of the present invention will become apparent from the following detailed description taken with the accompanying drawing.

### BRIEF DESCRIPTION OF THE DRAWINGS

FIG. 1 is a perspective view of an aerosolization device of the present invention;

FIG. 2 is an enlarged section view of the aerosolization device;

FIG. 3 is an enlarged view of Section A of FIG. 2;

FIG. 4 is a schematic view of an internal structure of the aerosolization device;

FIG. 5 is a flow chart of controlling the aerosolization device; and

FIG. 6 is an enlarged section view of an aperture plate in the aerosolization device.

### DETAILED DESCRIPTION OF THE INVENTION

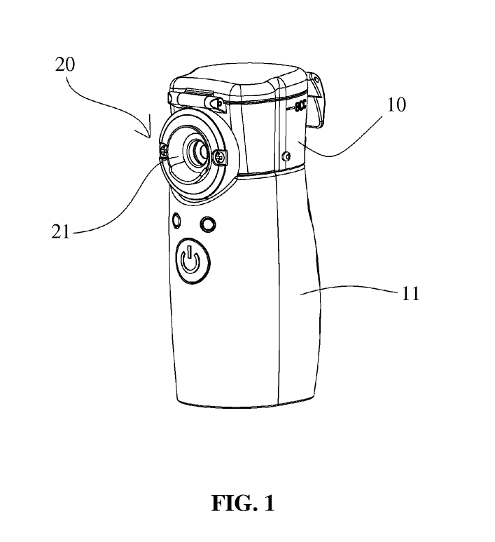

Refer to FIGS. 1 to 4, and aerosolization device of the invention comprises: a container 10 for containing a liquid 25, wherein the liquid 25 is a nebulizer medication; an oscillating aerosol generator 20 being installed at a position proximate to an opening at the bottom of the container 10, and interconnected with the container 10, for aerosolizing and spraying the liquid 25 supplied in the container 10 to the outside; a sensor 30 being disposed at a position close to the oscillating aerosol generator 20 at the bottom of the container 10 for detecting an oscillating signal from the oscillating aerosol generator 20 through the liquid 25, and an oscillating signal variation according to a condition whether or not the liquid 25 in the container 10 being in contact with the oscillating aerosol generator 20; a controller 40 for controlling the operation status of the oscillating aerosol generator 20 according to the oscillating signal variation detected by the sensor 30; and an alarm device 50 being electrically connected with the controller 40 and for sending out an alarm signal; wherein the liquid 25 is a medium for delivering the oscillating signal from the oscillating aerosol generator 20 to the sensor 30. In other words, the sensor 30 detects and monitors whether or not the liquid 25 in the container 10 is in contact with the oscillating aerosol generator 20 at anytime. If the supply of the liquid 25 is interrupted or exhausted, the controller 40 will control the oscillating aerosol generator 20 to stop its operations, so as to assure the aerosolization device could steadily supply a fixed quantity of aerosol medication to the patient in normal operation.

In FIG. 1, the container 10 includes a handle 11 disposed at the bottom of the container 10, and provided for a patient to hold the aerosolizaiton device.

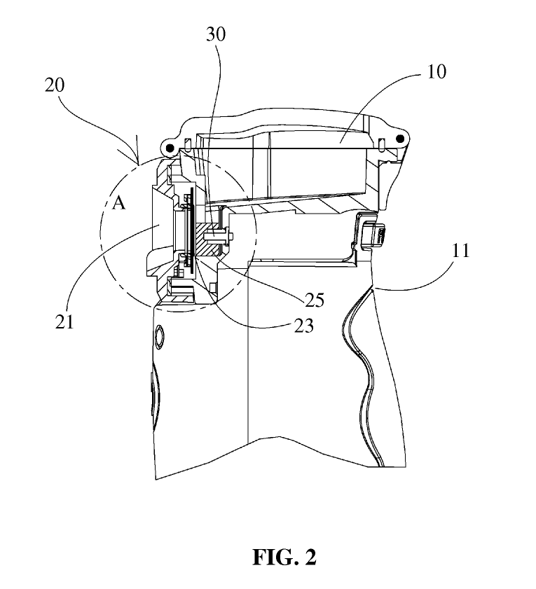

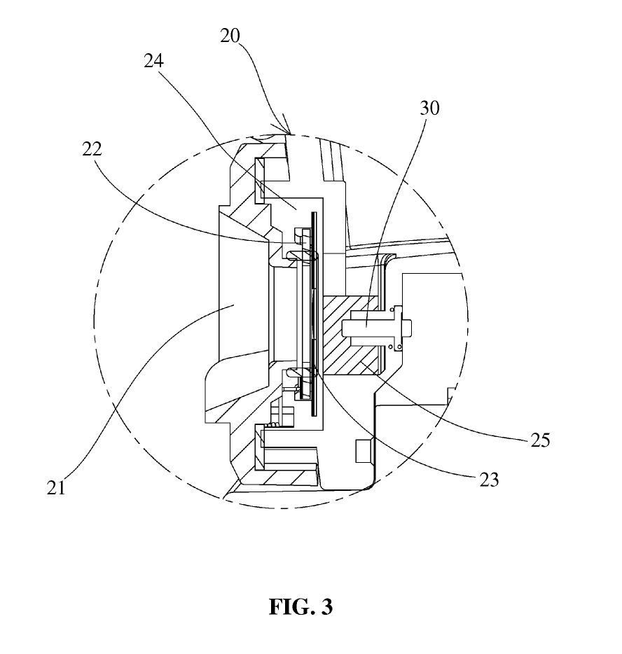

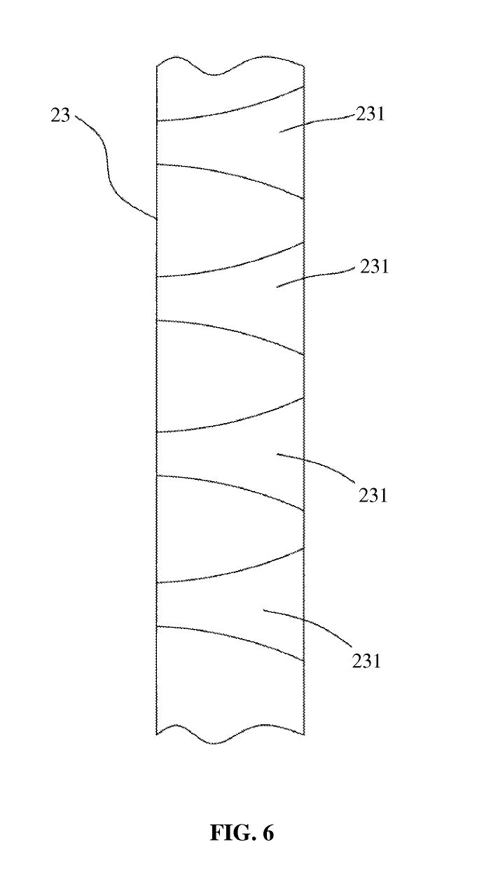

In FIGS. 2 and 3, the oscillating aerosol generator 20 is installed at a position proximate to an opening 12 at the bottom of the container 10, such that the liquid 25 can be aerosolized and sprayed from a nozzle 21 at a protective cover of the oscillating aerosol generator 20 to the outside. The oscillating aerosol generator 20 comprises an oscillator 22 and an aperture plate 23. The oscillator 22 is a piezoelectric element controlled by a controller 40 for supplying the operating voltage to the oscillator 22 to perform vibrations or stop operating. The aperture plate 23 comprises a plurality of conical aperture 231 having a small diameter and tapered from the container 10 to the outside (as shown in FIG. 6), and an elastic wrapping element 24 for wrapping the oscillator 22 and the aperture plate 23. If the oscillator 22 is vibrating, the aperture plate 23 will be driven to vibrate back and forth, and the back and forth vibrations of the aperture plate 23 driven to vibrate back and forth, and the back and forth vibrations of the aperture plate 23 drives the plurality of tapered conical apertures 231 to squeeze the liquid 25 in the container 10 to produced an aerosolization, and spray the aerosolized liquid 25 from the nozzle 21 of the protective cover.

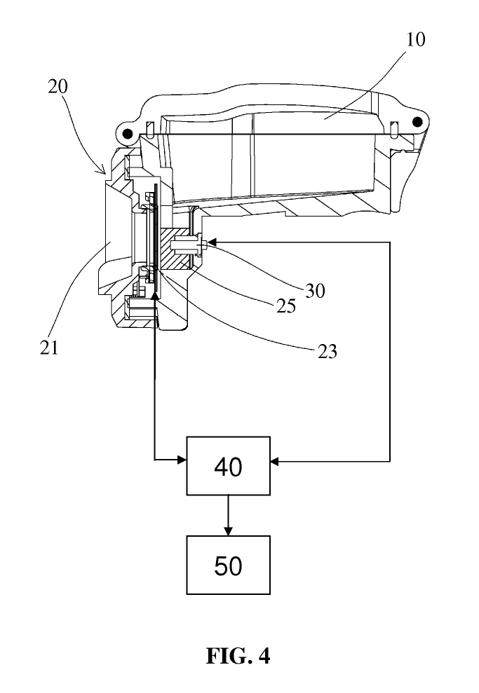

In FIG. 4, the sensor 30 is a conductive sensor for detecting an oscillating signal from the oscillating aerosol generator 20 through the liquid 25, and an oscillating signal variation according to a condition whether or not the liquid in the container 10 is in contact with oscillating aerosol generator 20. The liquid 25 is a medium for delivering the oscillating signal from the oscillating aerosol generator 20 to the sensor 30.

The controller 40 can receive the value of the vibration of oscillating signal detected by the sensor 30 for computation and comparison with a predetermined safe range. If the value of the detected variation exceeds the safe range, the controller 40 will immediately instruct the oscillating aerosol generator 20 to stop its operation, and simultaneously control the alarm device 50 to send out an alarm signal, such as a light or a sound.

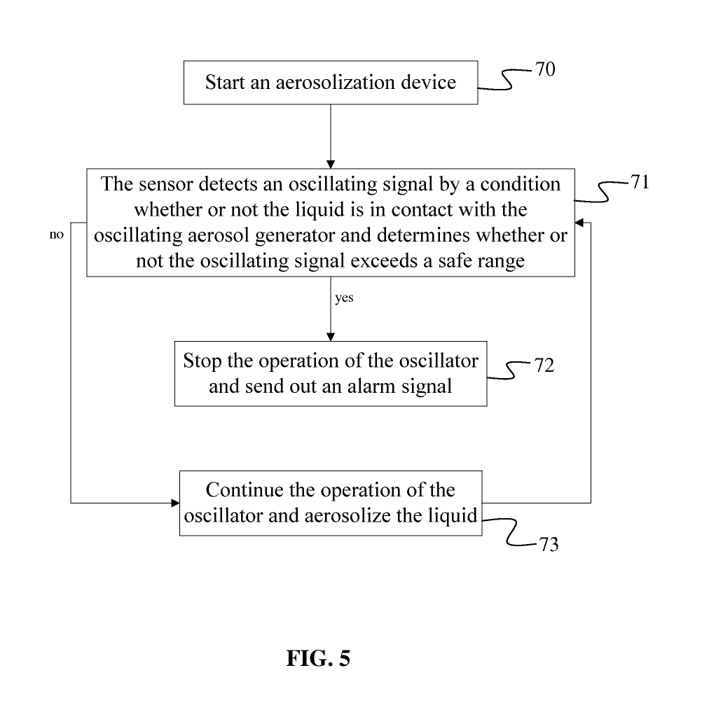

In FIG. 5, the procedure for controlling and operating the aforementioned aerosolization device comprises the following steps:

First step 70: Start an aerosolization device, and its operation will start according to a starting device pressed by a user.

Second step 71: A sensor detects an oscillating signal produced by condition whether or not the liquid is in contact with an oscillating aerosol generator, and determines whether or not the oscillating signal exceeds a safe range; if yes, then go to the fourth step 73, or else go to the third step 72.

Third step 72: continue the operation of the oscillator and aerosolize the liquid, and then return to second step 71 to continue the detection.

Fourth step 73: If the sensor detects that the liquid exceeds the predetermined safe range, the sensor will stop the operation of the oscillator immediately, and an alarm device will send out an alarm signal.

The aerosolization device of the invention uses the sensor to monitor whether or not the liquid in the container is in contact with the oscillating aerosol generator. If the supply of the liquid is interrupted or exhausted, the controller controls the oscillating aerosol generator to stop its operation immediately, so as to assure the normal operations of the aerosolizaiton device and steadily supply a fixed quantity of aerosol medication to patients.

By the detailed description of the overall structure and technical content of the present invention, the following advantages of the present invention can be derived: when the supply of the liquid is interrupted or exhausted, the controller will control the oscillating aerosol generator to stop its operation immediately and start an alarm device to send out an alarm signal at the same time to notice a patient to stop using the aerosolization device or refill the medication. Comparing to the prior art documents of U.S. Pat. No. 6,679,436 and U.S. Pat. No. 6,769,626, the aerosolization device of the present invention can shut down automatically when no liquid is in the container or the liquid in the container is exhausted, so as to achieve the effect of power-saving. For example, when the sensor detects that no liquid is in the container or the liquid in the container is exhausted, the controller stops the operation of the oscillating aerosol generator, controls the alarm device to send out said alarm signal, and then commands the aerosolization device shut down automatically.

While the invention has been described by way of examples and in terms of preferred embodiments, it is to be understood that the inventions is not limited thereto. To the contrary, it is intended to cover various modifications and similar arrangements and procedures, and the scope of the appended claims therefore should be accorded the broadest interpretation so as to encompass all such modifications and similar arrangements and procedures.

What is claimed is:

1. An aerosolization device comprising:
a container for containing a liquid;
an oscillating aerosol generator being installed at a position proximate to an opening at the bottom of said container for aerosolizing and spraying said liquid in said container to the outside;
a sensor being disposed at a position close to said oscillating aerosol generator at the bottom of said container for detecting an oscillating signal from said oscillating aerosol generator through said liquid, and an oscillating signal variation according to a condition whether or not said liquid in said container is in contact with said oscillating aerosol generator; and
a controller for controlling the operation status of said oscillating aerosol generator according to said oscillating signal variation detected by said sensor,
wherein said liquid is a medium for delivering said oscillating signal from said oscillating aerosol generator to said sensor.

2. The aerosolization device according to claim 1, wherein said oscillating aerosol generator comprises an oscillator and an aperture plate.

3. The aerosolization device according to claim 2, wherein said aperture plate comprises a plurality of conical apertures tapered from said container to the outside.

4. The aerosolization device according to claim 2, wherein said oscillator comprises a piezoelectric element.

5. The aerosolization device according to claim 2, wherein said oscillator is provided for vibrating said aperture plate in said oscillating aerosol generator.

6. The aerosolization device according to claim 1, wherein said liquid comprises a nebulizer medication.

7. The aerosolization device according to claim 1, wherein said sensor comprises a conductive sensor.

8. The aerosolization device according to claim 1, wherein said controller presets a specific range for said oscillating signal variation.

9. The aerosolization device according to claim 8, further comprising an alarm device electrically connected with said controller and for sending out an alarm signal, wherein when said oscillating signal detected by the sensor exceeds said specific range, said controller stopping the operation of said oscillating aerosol generator and controlling said alarm device to send out said alarm signal synchronously.

10. The aerosolization device according to claim 9, wherein said alarm signal is selected from the group consisting of: a light signal and a sound signal.
